(19) United States
(12) Patent Application Publication
(10) Pub. No.: US 2025/0107721 A1
(43) Pub. Date: Apr. 3, 2025

(54) PULSE PRESSURE MEASURING APPARATUS

(71) Applicant: GUANGZHOU LUXVISIONS INNOVATION TECHNOLOGY LIMITED, GUANGZHOU (CN)

(72) Inventors: Chih-Ju Lin, Guangzhou (CN); Shih-Chieh Yen, GUANGZHOU (CN); Yi-Wei Liu, Guangzhou (CN); Wei-Han Wu, Guangzhou (CN)

(73) Assignee: GUANGZHOU LUXVISIONS INNOVATION TECHNOLOGY LIMITED, GUANGZHOU (CN)

(21) Appl. No.: 18/621,060

(22) Filed: Mar. 28, 2024

Related U.S. Application Data
(60) Provisional application No. 63/587,156, filed on Oct. 2, 2023.

(30) Foreign Application Priority Data
Feb. 1, 2024 (CN) 202410142705.0

(51) Int. Cl. A61B 5/024

(52) U.S. Cl. CPC A61B 5/02444; A61B 5/02438; A61B 2562/0247; A61B 2562/046

(57) ABSTRACT

A pulse pressure measuring apparatus including a plurality of pressing elements, a plurality of pressure sensors, and a processing unit is provided. The pressing elements are used to press the site to be measured, and each pressing element has a position coordinate Pᵢ (i=1, 2, 3 . . . ). The pressure sensors are configured to respectively measure pressure on the pressing elements to generate measured values of pressure intensity Iᵢ (i=1, 2, 3 . . . ) at the position coordinates Pᵢ (i=1, 2, 3 . . . ). The processing unit utilizes the position coordinates Pᵢ (i=1, 2, 3 . . . ) and the measured values of pressure intensity Iᵢ (i=1, 2, 3 . . . ) to determine the blood vessel locus.

### CROSS-REFERENCE TO RELATED APPLICATION

This application claims the priority benefit of U.S. provisional application Ser. No. 63/587,156, filed on Oct. 2, 2023 and China application serial no. 202410142705.0, filed on Feb. 1, 2024. The entirety of each of the above-mentioned patent applications is hereby incorporated by reference herein and made a part of this specification.

### BACKGROUND

### Technical Field

The invention relates to the pulse pressure measuring apparatus.

### Description of Related Art

In traditional Chinese medicine, the doctor puts his/her fingers on the patient's wrist and applies pressure to sense pulse wave changes, and then considers all the information to complete the diagnosis. However, doctors often have different guidelines for diagnosis, all of these are based on their personal diagnosis and treatment experience. Therefore, an objective and quantifiable pulse pressure measuring apparatus is needed. The pulse pressure measuring apparatuses on the present market do not consider the locus of blood vessel, it caused that the results in low accuracy of pulse measurement.

The information disclosed in this Background section is only for enhancement of understanding of the background of the described technology and therefore it may contain information that does not form the prior art that is already known to a person of ordinary skill in the art. Further, the information disclosed in the Background section does not mean that one or more problems to be resolved by one or more embodiments of the invention was acknowledged by a person of ordinary skill in the art.

### SUMMARY

The present invention provides a pulse pressure measuring apparatus that determines blood vessel locus and has good measurement accuracy.

According to an embodiment of the present invention, a pulse pressure measuring apparatus is provided. The pulse pressure measuring apparatus includes a base, a plurality of pressing elements, a plurality of pressure sensors and a processing unit. The pressing elements are arranged in an array on the base and are used to press the site to be measured, wherein the number of the pressing elements is at least four, and each pressing element has a position coordinate Pᵢ (i=1, 2, 3 . . . ). The pressure sensors are configured to respectively measure pressure on the pressing elements to generate measured values of pressure intensity Iᵢ (i=1, 2, 3 . . . ) at the position coordinates Pᵢ (i=1, 2, 3 . . . ). The processing unit is connected to the pressing elements and the pressure sensors. In the blood vessel locus determination stage, the processing unit utilizes any three of the position coordinates Pᵢ (i=1, 2, 3 . . . ) and the corresponding three measured values of pressure intensity Iᵢ, Iⱼ, Iₖ (i, j, k=1, 2, 3 . . . and i≠j≠k) to produce multiple weighted coordinate positions Gᵢⱼₖ, wherein

$${G}_{ijk} ( X , Y ) = \frac{{I}_{i} {P}_{i} + {I}_{j} {P}_{j} + {I}_{k} {P}_{k}}{{I}_{i} + {I}_{j} + {I}_{k}}$$

The processing unit defines multiple linear equations respectively passing through the weighted coordinate positions Gᵢⱼₖ, and determines the blood vessel locus of the site to be measured based on the band defined by the linear equations and the barycentric coordinate of the weighted coordinate positions Gᵢⱼₖ.

According to an embodiment of the present invention, a pulse pressure measuring apparatus is provided. The pulse pressure measuring apparatus includes a base, a plurality of pressing elements, a plurality of pressure sensors and a processing unit. The pressing elements are arranged in an array on the base and are used to press the site to be measured, wherein the number of the pressing elements is at least four, and each pressing element has a position coordinate Pᵢ (i=1, 2, 3 . . . ). The pressure sensors are configured to respectively measure pressure on the pressing elements to generate measured values of pressure intensity Iᵢ (i=1, 2, 3 . . . ) at the position coordinates Pᵢ (i=1, 2, 3 . . . ). The processing unit is connected to the pressing elements and the pressure sensors. In the blood vessel locus determination stage, the processing unit defines multiple virtual circles, wherein the position coordinates Pᵢ (i=1, 2, 3 . . . ) are respectively centers of the virtual circles and reciprocals of the square root of the measured values of pressure intensity Iᵢ (i=1, 2, 3 . . . ) are respectively radii of the virtual circles. The processing unit further defines a plurality of interior common tangents of the virtual circles, shrinks or enlarges the virtual circles by a same ratio until at least two of the interior common tangents overlap with each other, and defines the blood vessel locus of the site to be measured based on the interior common tangents overlapping with each other.

Based on the above description, the pulse pressure measuring apparatus provided in the embodiments of the present invention utilizes multiple pressing elements and multiple pressure sensors to obtain the blood vessel locus. Compared with the traditional pulse pressure measuring apparatuses that do not consider the blood vessel locus, the pulse pressure measuring apparatus provided in the embodiments of the present invention may more accurately measure the pulse wave of the site to be measured.

Other objectives, features and advantages of the present invention will be further understood from the further technological features disclosed by the embodiments of the present invention wherein there are shown and described preferred embodiments of this invention, simply by way of illustration of modes best suited to carry out the invention.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a schematic diagram of the pulse pressure measuring apparatus according to an embodiment of the present invention.

FIG. 1B is a block diagram of the pulse pressure measuring apparatus according to an embodiment of the present invention.

FIG. 1C is a schematic diagram of the usage of the pulse pressure measuring apparatus according to an embodiment of the present invention.

FIG. 2A is a schematic diagram of the pulse pressure measuring apparatus according to an embodiment of the present invention.

FIG. 2B is a schematic diagram of a determination method for blood vessel locus according to the first embodiment of the present invention.

FIG. 3A to FIG. 3E are schematic diagrams of a determination method for blood vessel locus according to the second embodiment of the present invention.

FIG. 4 is a schematic diagram of the pulse pressure measuring apparatus according to an embodiment of the present invention.

FIG. 5 is a schematic diagram of a pulse wave optimization method according to an embodiment of the present invention.

FIG. 6A, FIG. 6B, FIG. 7A, FIG. 7B, FIG. 7C, and FIG. 8 are schematic diagrams of the pulse pressure measuring apparatus according to embodiments of the present invention.

### DESCRIPTION OF THE EMBODIMENTS

In the following detailed description of the preferred embodiments, reference is made to the accompanying drawings which form a part hereof, and in which are shown by way of illustration specific embodiments in which the invention may be practiced. In this regard, directional terminology, such as “top,” “bottom,” “front,” “back,” etc., is used with reference to the orientation of the Figure(s) being described. The components of the present invention can be positioned in a number of different orientations. As such, the directional terminology is used for purposes of illustration and is in no way limiting. On the other hand, the drawings are only schematic and the sizes of components may be exaggerated for clarity. It is to be understood that other embodiments may be utilized and structural changes may be made without departing from the scope of the present invention. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings. Similarly, the terms “facing,” “faces” and variations thereof herein are used broadly and encompass direct and indirect facing, and “adjacent to” and variations thereof herein are used broadly and encompass directly and indirectly “adjacent to”. Therefore, the description of “A” component facing “B” component herein may contain the situations that “A” component directly faces “B” component or one or more additional components are between “A” component and “B” component. Also, the description of “A” component “adjacent to” “B” component herein may contain the situations that “A” component is directly “adjacent to” “B” component or one or more additional components are between “A” component and “B” component. Accordingly, the drawings and descriptions will be regarded as illustrative in nature and not as restrictive.

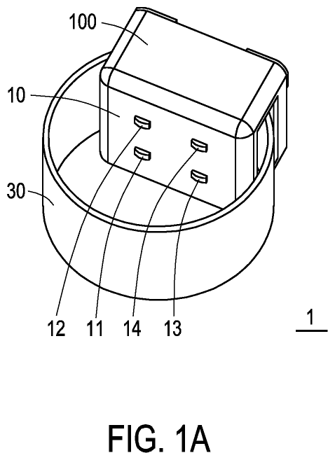

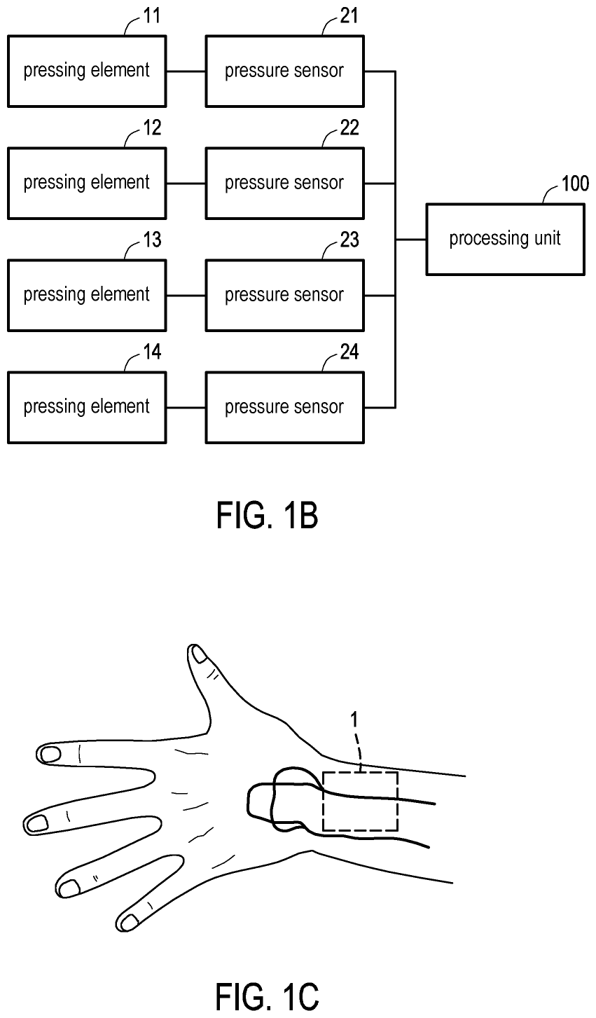

Please Refer to FIG. 1A to FIG. 1C. The pulse pressure measuring apparatus 1 according to an embodiment of the present invention is provided. The pulse pressure measuring apparatus 1 includes a base 10, a plurality of pressing elements 11, 12, 13, 14 arranged in an array on the base 10, a plurality of pressure sensors 21, 22, 23, 24 accommodated inside the base 10, and a processing unit 100. The pressing elements 11, 12, 13, 14 are used to press the site to be measured. In some embodiments, the pulse pressure measuring apparatus 1 may be worn on the hand of the user by the fixing member 30 as shown in FIG. 1C, and the site to be measured is the wrist, but the present invention is not limited thereto. The pressure sensors 21, 22, 23, and 24 are used to measure pressure on the pressing elements 11, 12, 13, and 14, respectively. The processing unit 100 connects the pressing elements 11, 12, 13, 14 and the pressure sensors 21, 22, 23, 24 so as to control the pressing elements 11, 12, 13, 14 to perform a pressing program, and to read measurement data of the pressure sensors 21, 22, 23, 24. It should be noted that, for the convenience of understanding, in the embodiment shown in FIGS. 1A to 1C, the number of the pressing elements 11, 12, 13, 14 and the number of the pressure sensors 21, 22, 23, 24 are both 4. However, the present invention is not limited thereto. In various embodiments of the present invention, the number of pressing elements and the number of pressure sensors may be equal to 4 or greater than 4.

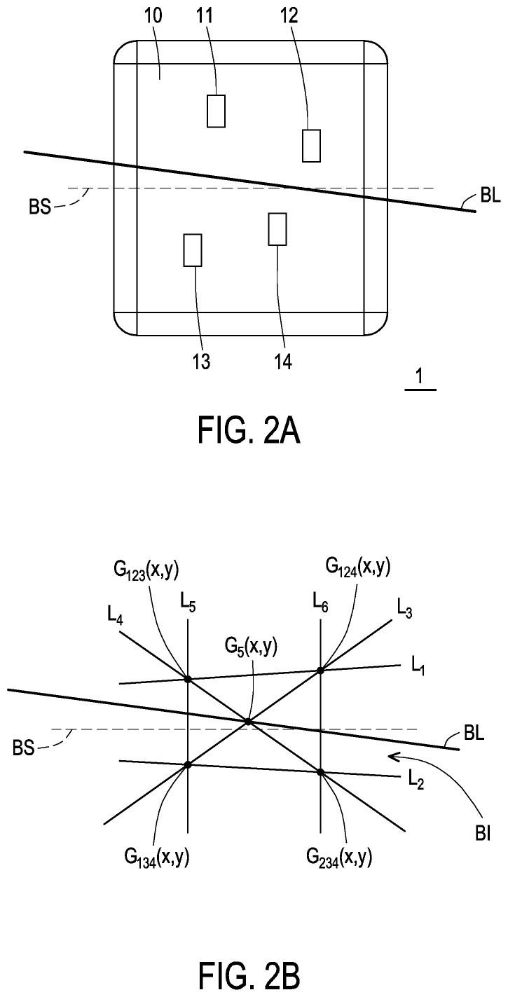

Next, please refer to FIG. 1A, FIG. 1B, FIG. 2A and FIG. 2B. A determination method for blood vessel locus is provided according to the first embodiment of the present invention.

In the first embodiment, the pressing elements 11, 12, 13, and 14 are respectively disposed at different position coordinates P₁ (x,y), P₂ (x,y), P₃ (x,y), P₄ (x,y). In the blood vessel locus determination stage, the pressure sensors 21, 22, 23, 24 respectively obtain the measured values of pressure intensity I₁, I₂, I₃, I₄ due to the pressing programs performed by the pressing elements 11, 12, 13, 14. The measured values of pressure intensity I₁, I₂, I₃, I₄ respectively correspond to the position coordinates P₁ (x,y), P₂ (x,y), P₃ (x,y), and P₄ (x,y).

Next, based on the principle that the intensity of the sensing signal would decrease as the distance between the signal source and the sensor increases, the processing unit 100 utilizes any three of the position coordinates P₁ (x,y), P₂ (x,y), P₃ (x,y), P₄ (x,y) and the corresponding three of the measured values of pressure intensity I₁, I₂, I₃, I₄ to generate multiple weighted coordinate positions G₁₂₃ (x,y), G₁₂₄ (x,y), G₁₃₄ (x,y), G₂₃₄ (x,y), where P₁, P₂, P₃, P₄ respectively represent P₁ (x,y), P₂ (x,y), P₃ (x,y), P₄ (x,y):

$${G}_{123} ( x , y ) = \frac{{I}_{1} {P}_{1} + {I}_{2} {P}_{2} + {I}_{3} {P}_{3}}{{I}_{1} + {I}_{2} + {I}_{3}} ;$$

$${G}_{124} ( x , y ) = \frac{{I}_{1} {P}_{1} + {I}_{2} {P}_{2} + {I}_{4} {P}_{4}}{{I}_{1} + {I}_{2} + {I}_{4}} ;$$

$${G}_{134} ( x , y ) = \frac{{I}_{1} {P}_{1} + {I}_{3} {P}_{3} + {I}_{4} {P}_{4}}{{I}_{1} + {I}_{3} + {I}_{4}} ;$$

$${G}_{234} ( x , y ) = \frac{{I}_{2} {P}_{2} + {I}_{3} {P}_{3} + {I}_{4} {P}_{4}}{{I}_{2} + {I}_{3} + {I}_{4}} .$$

Then, the processing unit 100 defines a plurality of linear equations respectively passing through the weighted coordinate positions G₁₂₃ (x,y), G₁₂₄ (x,y), G₁₃₄ (x,y), G₂₃₄ (x,y). These linear equations are shown as the straight line L₁˜L₆ in FIG. 2B, where the straight line L₁ passes through the weighted coordinate positions G₁₂₃ (x,y) and G₁₂₄ (x,y), the straight line L₂ passes through the weighted coordinate positions G₁₃₄ (x,y) and G₂₃₄ (x,y), the straight line L₃ passes through the weighted coordinate positions G₁₂₄ (x,y) and G₁₃₄ (x,y), the straight line L₄ passes through the weighted coordinate positions G₁₂₃ (x,y) and G₂₃₄ (x,y), the straight line L₅ passes through the weighted coordinate positions G₁₂₃ (x,y) and G₁₃₄ (x,y), and the straight line L₆ passes through the weighted coordinate positions G₁₂₄ (x,y) and G₂₃₄ (x,y).

Further, based on the wearing orientation of the pulse pressure measuring apparatus 1 and a reasonable estimation for the direction of the blood vessel locus of the site to be measured, the processing unit 100 utilizes the straight lines (i.e., the straight lines L₁, L₂, L₃, L₄, as shown in FIG. 2B), among the straight lines L₁ to L₆, having a slope within ±0.5 relative to the baseline BS to define a band, wherein the baseline BS is the central symmetry axis of the pulse pressure measuring apparatus 1 (as shown in FIG. 2A), and is defined as a straight line having a slope of zero. Specifically, as shown in FIG. 2B, the straight lines L₁ and L₂ are used to define the band BI. It should be noted that although the straight lines L₅ and L₆ also define another band as shown in FIG. 2B, it is reasonably inferred that the blood vessel locus of the site to be measured would not be situated within the band defined by the straight lines L₅, L₆ in consideration of the wearing orientation of the pulse pressure measuring apparatus 1. Therefore, the slope of the straight lines used to define the band is limited to ±0.5 relative to the baseline BS, as mentioned above.

Subsequently, the processing unit 100 produces the barycentric coordinate G₅ (x,y) of the weighted coordinate positions G₁₂₃ (x,y), G₁₂₄ (x,y), G₁₃₄ (x,y), G₂₃₄ (x,y) by calculation, wherein the x coordinate of the barycentric coordinate G₅ (x,y) is the average value of the x coordinates of the weighted coordinate positions G₁₂₃ (x,y), G₁₂₄ (x,y), G₁₃₄ (x,y), G₂₃₄ (x,y), and the y coordinate of the barycentric coordinate G₅ (x,y) is the average value of the y coordinates of the weighted coordinate positions G₁₂₃ (x,y), G₁₂₄ (x,y), G₁₃₄ (x,y), G₂₃₄ (x,y).

Then, the processing unit 100 determines the blood vessel locus BL of the site to be measured based on the average value of the slope of both the straight lines L₁ and L₂ and the barycentric coordinate G₅ (x,y), wherein the slope of the blood vessel locus BL is the average value of the slope of both the straight lines L₁ and L₂, and the blood vessel locus BL passes through the barycentric coordinate G₅ (x,y). It should be noted that the blood vessel locus BL mentioned above is not the locus of the real blood vessel of the site to be measured. Specifically, the blood vessel locus BL mentioned above corresponds to the locus of the real blood vessel and is located on the plane where the pressing elements 11, 12, 13, 14 are located. Compared with the traditional pulse pressure measuring apparatuses that do not consider the blood vessel locus, the pulse pressure measuring apparatus 1 provided according to the embodiment of the present invention utilizes the pressing elements 11, 12, 13, 14 and the pressure sensors 21, 22, 23, 24 to obtain the blood vessel locus BL. Accordingly, the pulse wave of the site to be measured may be more precisely measured.

In some embodiments, in order to improve the accuracy of the above-mentioned blood vessel locus determination stage and reduce the interference of noise, the spacings between adjacent ones of the pressing elements 11, 12, 13, 14 are less than or equal to 30 mm in the direction parallel to the baseline BS and in the direction perpendicular to the baseline BS. In this manner, the intensity attenuation of the pulse pressure, which reduces the measurement accuracy, resulted from long transmission distance of the pulse wave and the interference of noise may be avoided.

In some embodiments, in the above-mentioned blood vessel locus determination stage, the pressing elements 11, 12, 13, 14 simultaneously press the site to be measured to generate pulse pressure having higher intensity, thereby improving the measurement accuracy of the pulse pressure measuring apparatus 1.

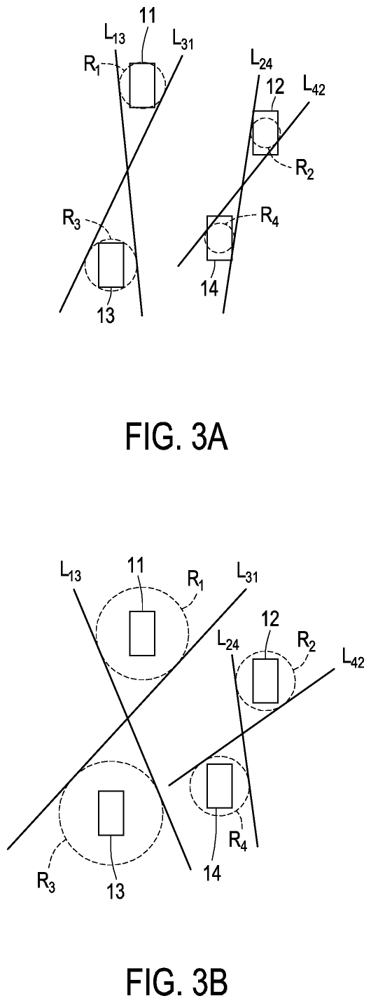

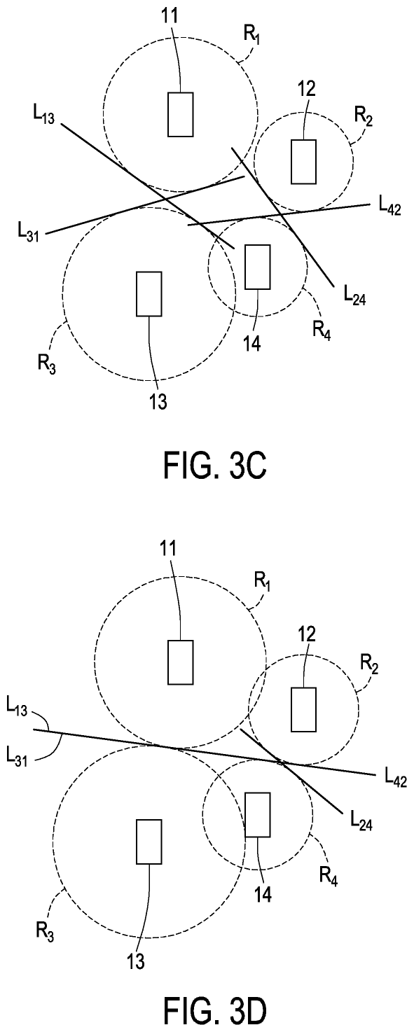

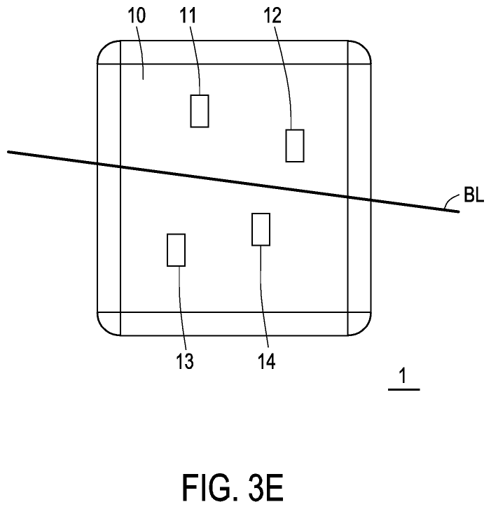

Next, please refer to FIG. 1A, FIG. 1B, FIG. 3A, FIG. 3B, FIG. 3C, FIG. 3D and FIG. 3E. According to the second embodiment of the present invention, another determination method for blood vessel locus is provided hereinafter.

In the second embodiment, the pressing elements 11, 12, 13, 14 are respectively disposed at the position coordinates P₁ (x,y), P₂ (x,y), P₃ (x,y), P₄ (x,y) on the base 10. In the blood vessel locus determination stage, the pressure sensors 21, 22, 23, 24 respectively obtain the measured values of pressure intensity I₁, I₂, I₃, I₄ due to the pressing programs performed by the pressing elements 11, 12, 13, 14. The measured values of pressure intensity I₁, I₂, I₃, I₄ respectively correspond to the position coordinates P₁ (x,y), P₂ (x,y), P₃ (x,y), P₄ (x,y).

Next, based on the principle that the intensity of the sensing signal would be inversely proportional to the square of the distance between the signal source and the sensor, it may be known that the distance between the signal source and the sensor would be inversely proportional to the square root of the intensity. Furthermore, based on the wearing orientation of the pulse pressure measuring apparatus 1, it may be known that the pressing elements 11 and 13 would correspond to a same pulse wave source, and the pressing elements 12 and 14 would correspond to a same pulse wave source. It may be inferred that the distance between the pressing element 11 and the corresponding pulse wave source and the distance between the pressing element 13 and the corresponding pulse wave source have a ratio of 1/(I₁)^{1/2}:1/(I₃)^{1/2}, and the distance between the pressing element 12 and the corresponding pulse wave source and the distance between the pressing element 14 and the corresponding pulse wave source have a ratio of 1/(I₂)^{1/2}:1/(I₄)^{1/2}. Accordingly, the processing unit 100 defines a virtual circle R₁ having a center at the position coordinate P₁ (x,y) and a radius of 1/(I₁)^{1/2}, a virtual circle R₃ having a center at the position coordinate P₃ (x,y) and a radius of 1/(I₃)^{1/2}, and the interior common tangents L₁₃ and L₃₁ of the virtual circles R₁ and R₃, as shown in FIG. 3A. Similarly, the processing unit 100 defines a virtual circle R₂ having a center at the position coordinate P₂ (x,y) and a radius of 1/(I₂)^{1/2}, a virtual circle R₄ having a center at the position coordinate P₄ (x,y) and a radius of 1/(I₄)^{1/2}, and the interior common tangents L₂₄ and L₄₂ of the virtual circles R₂ and R₄, as shown in FIG. 3A. The interior common tangent L₁₃ and the interior common tangent L₃₁ correspond to a same pulse wave source, and the interior common tangent L₂₄ and the interior common tangent L₄₂ correspond to a same pulse wave source.

Then, referring to FIG. 3A to FIG. 3D, the processing unit 100 enlarges (or shrinks) the virtual circles R₁, R₂, R₃, R₄ by a same ratio until the interior common tangents corresponding to different pulse wave sources overlap with each other.

As specifically shown in FIG. 3D, the interior common tangent L₁₃ (and the interior common tangent L₃₁) overlaps with the interior common tangent L₄₂, wherein the interior common tangent L₁₃ (and the interior common tangent L₃₁) and the interior common tangent L₄₂ correspond to different pulse wave sources. In this manner, the radius of the virtual circle R₁ is C1 times 1/(I₁)^{1/2} (i.e., C1/(I₁)^{1/2}), the radius of the virtual circle R₂ is C1 times 1/(I₂)^{1/2} (i.e., C1/(I₂)^{1/2}), the radius of virtual circle R₃ is C1 times 1/(I₃)^{1/2} (i.e., C1/(I₃)^{1/2}), and the radius of virtual circle R₄ is C1 times 1/(I₄)^{1/2} (i.e., C1/(I₄)^{1/2}), wherein the scale C1 is any number greater than 0.

Finally, the processing unit 100 defines the straight line defined by the overlapping interior common tangents as the blood vessel locus BL, as shown in FIG. 3D and FIG. 3E. It should be noted that the blood vessel locus BL mentioned above is not the locus of the real blood vessel of the site to be measured, but corresponds to the locus of the real blood vessel. Specifically, the blood vessel locus BL mentioned above is located on the plane where the pressing elements 11, 12, 13, 14 are located. Compared with the traditional pulse pressure measuring apparatuses that do not consider the blood vessel locus, the pulse pressure measuring apparatus 1 provided according to the embodiment of the present invention utilizes the pressing elements 11, 12, 13, 14 and the pressure sensors 21, 22, 23, 24 to obtain the blood vessel locus BL. Accordingly, the pulse wave of the site to be measured may be more precisely measured.

In some embodiments, in order to improve the accuracy of the above-mentioned blood vessel locus determination stage and reduce the interference of noise, the spacings between adjacent ones of the pressing elements 11, 12, 13, 14 are less than or equal to 30 mm in the direction parallel to the baseline BS and in the direction perpendicular to the baseline BS. In this manner, the intensity attenuation of the pulse pressure, which reduces the measurement accuracy, resulted from long transmission distance of the pulse wave and the interference of noise may be avoided.

In some embodiments, in the above-mentioned blood vessel locus determination stage, the pressing elements 11, 12, 13, 14 simultaneously press the site to be measured to generate pulse pressure having higher intensity, thereby improving the measurement accuracy of the pulse pressure measuring apparatus 1.

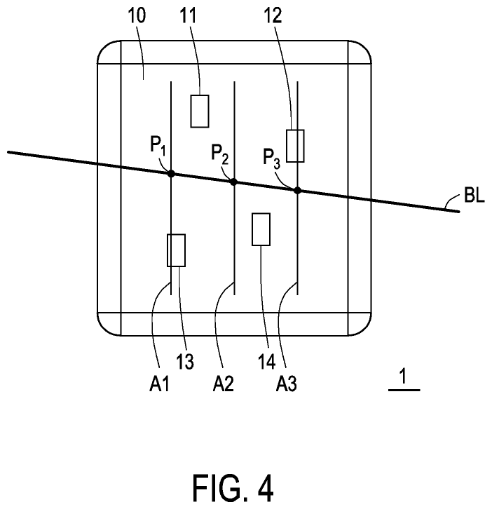

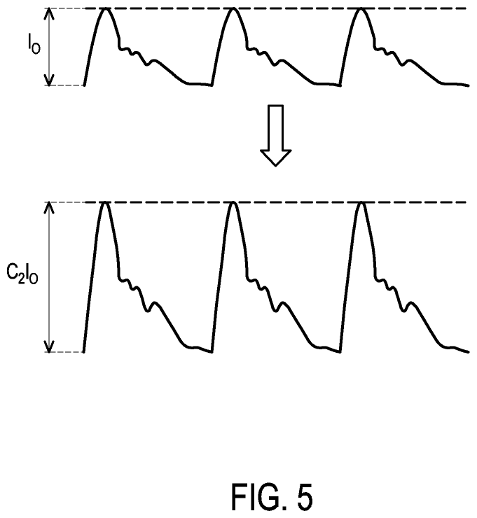

Please refer to FIG. 4 and FIG. 5. when the pulse pressure measuring apparatus 1 is worn on the site to be measured, the processing unit 100 may define multiple virtual lines A1, A2, A3 according to the built-in database of the processing unit 100 or the user's settings. The points P₁, P₂, P₃ where the virtual lines A1, A2, A3 intersect with the blood vessel locus BL are used as points corresponding to the pulse wave sources of the site to be measured. In some embodiments, the site to be measured is the wrist of the user. Based on the three pulse wave sources of the wrist, the virtual lines A1, A2, A3 are three parallel lines having the spacing of 1.0 cm to 1.2 cm therebetween, but the invention is not limited thereto. In some embodiments, the site to be measured is another part of the human body, the number of virtual lines is not limited to 3, and the spacings between the multiple virtual lines are not limited. During the pulse wave measurement stage of the pulse pressure measuring apparatus 1, based on the positions of the pressing elements 11, 12, 13, 14 and the positions of the points P₁, P₂, P₃, the processing unit 100 would determine which pressing element and corresponding pressure sensor should be used for a specific pulse wave source.

In one embodiment, the processing unit 100 selects the pressing element 12 and the pressure sensor 22 to measure the pulse wave source corresponding to the point P₃, and hence obtains the pulse wave diagram (FIG. 5), wherein the amplitude is shown as I₀. Furthermore, the processing unit 100 takes I₀ times a scale C2 as the actual amplitude of the pulse wave generated by the pulse wave source, wherein the scale C2 is the square root of the ratio of the shortest distance between the pressing element 12 and the blood vessel locus BL and the scale CL.

In other embodiments, in which different one(s) of the pressing elements 11, 12, 13, 14 and the corresponding pressure sensors 21, 22, 23, 24 are used for the pulse wave measurement, based on the measured amplitude, a method similar to the above-mentioned method would be used to obtain the actual amplitude. For example, when the processing unit 100 selects the pressing element 14 and the pressure sensor 24 to measure the pulse wave source corresponding to the point P₂, and hence obtains the pulse wave diagram having the amplitude of I₀ (FIG. 5), the processing unit 100 would take I₀ times a scale C2 as the actual amplitude of the pulse wave generated by the pulse wave source, wherein the scale C2 is the square root of the ratio of the shortest distance between the pressing element 14 and the blood vessel locus BL and the scale CL.

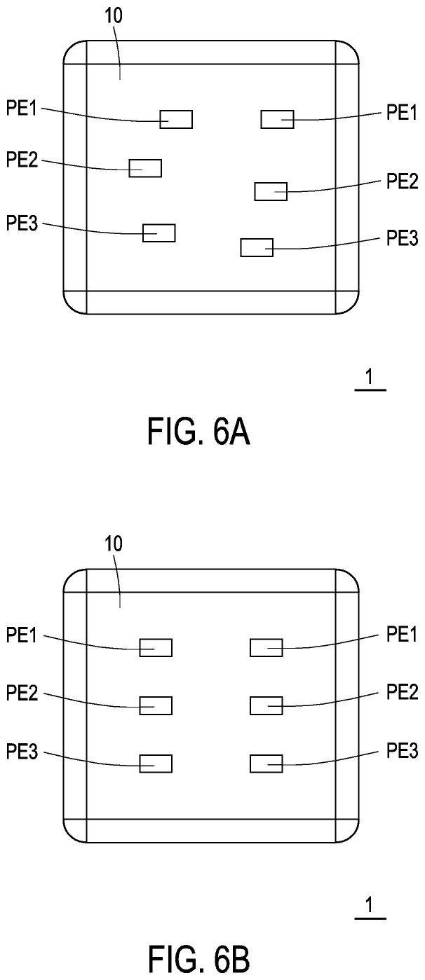

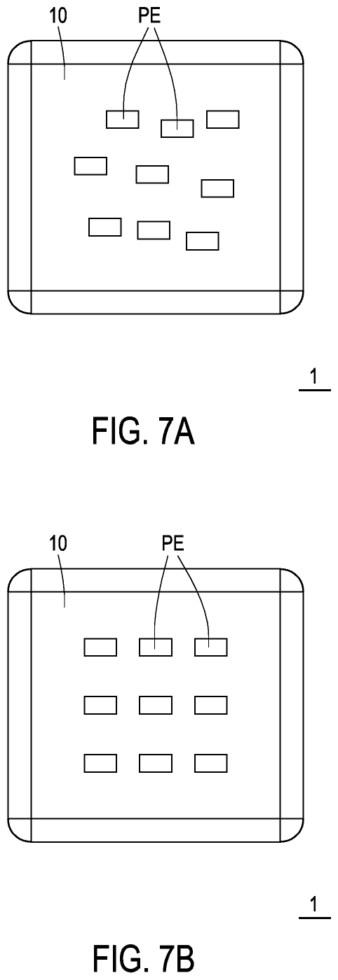

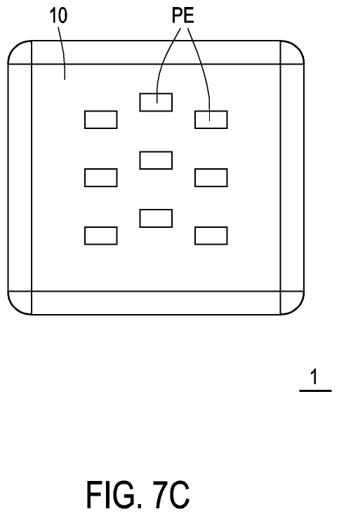

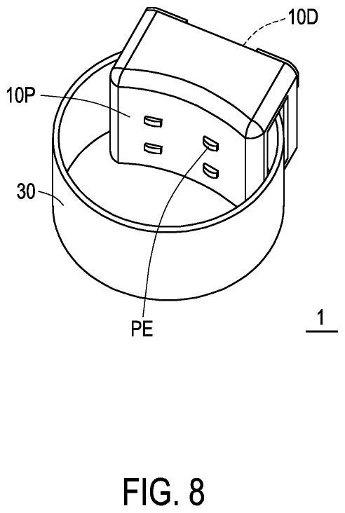

Please refer to FIG. 6A and FIG. 6B. In some embodiments, the pulse pressure measuring apparatus 1 may include two pressing elements PE1, two pressing elements PE2 and two pressing elements PE3. The above-mentioned six pressure sensors may be arranged in a misaligned manner (FIG. 6A) or in an aligned manner (FIG. 6B). A larger number of the pressure sensors may improve the measurement accuracy of the pulse pressure measuring apparatus 1. In the pulse wave measurement stage of the pulse pressure measuring apparatus 1, the two pressing elements PE1 arranged in pair, the two pressing elements PE2 arranged in pair, or the two pressing elements PE3 arranged in pair may simultaneously perform the pressing program with a same pressure value.

Please Refer to FIG. 7A, FIG. 7B and FIG. 7C. In some embodiments, the pulse pressure measuring apparatus 1 may include nine pressing elements PE. The nine pressure sensors may be irregularly arranged (FIG. 7A), neatly arranged (FIG. 7B), or regularly misaligned (FIG. 7C). In the pulse wave measurement stage of the pulse pressure measuring apparatus 1, the multiple pressing elements PE may simultaneously perform the pressing program with a same pressure value.

Please Refer to FIG. 8. In some embodiments, the pulse pressure measuring apparatus 1 may include a base 10P, and multiple pressing elements PE of the pulse pressure measuring apparatus 1 are arranged on the curved surface of the base 10P. The pulse pressure measuring apparatus 1 may also include a display screen 10D to display pulse pressure data.

To sum up, the pulse pressure measuring apparatus provided in the embodiments of the present invention utilizes multiple pressing elements and multiple pressure sensors to obtain the blood vessel locus. Compared with the traditional pulse pressure measuring apparatuses that do not consider the blood vessel locus, the pulse pressure measuring apparatus provided by the embodiments of the present invention may more accurately measure the pulse wave of the site to be measured.

The foregoing description of the preferred embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, and the reference to particularly preferred exemplary embodiments of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. The abstract of the disclosure is provided to comply with the rules requiring an abstract, which will allow a searcher to quickly ascertain the subject matter of the technical disclosure of any patent issued from this disclosure. It is submitted with the understanding that it will not be used to interpret or limit the scope or meaning of the claims. Any advantages and benefits described may not apply to all embodiments of the invention. It should be appreciated that variations may be made in the embodiments described by persons skilled in the art without departing from the scope of the present invention as defined by the following claims. Moreover, no element and component in the present disclosure is intended to be dedicated to the public regardless of whether the element or component is explicitly recited in the following claims.

What is claimed is:

1. A pulse pressure measuring apparatus, including:
a base;
a plurality of pressing elements, arranged in an array on the base and used to press a site to be measured, wherein a number of the pressing elements is at least four, and each of the pressing elements has a position coordinate Pᵢ (i=1, 2, 3 . . . );
a plurality of pressure sensors, configured to respectively measure pressure on the plurality of pressing elements in order to generate measured values of pressure intensity Iᵢ (i=1, 2, 3 . . . ) at the position coordinates Pᵢ (i=1, 2, 3 . . . ); and
a processing unit, connected to the plurality of pressing elements and the plurality of pressure sensors,
wherein, in a blood vessel locus determination stage, the processing unit utilizing any three of the plurality of position coordinates Pᵢ (i=1, 2, 3 . . . ) and the corresponding three measured values of pressure intensity Iᵢ, Iⱼ, Iₖ (i, j, k=1, 2, 3 . . . , and i≠j≠k) to generate multiple weighted coordinate positions Gᵢⱼₖ, where
$${G}_{ijk} ( X , Y ) = \frac{{I}_{i} {P}_{i} + {I}_{j} {P}_{j} + {I}_{k} {P}_{k}}{{I}_{i} + {I}_{j} + {I}_{k}} ,$$
wherein the processing unit defines a plurality of linear equations respectively passing through the plurality of weighted coordinate positions Gᵢⱼₖ, and determines a blood vessel locus of the site to be measured based on a band defined by the linear equations and a barycentric coordinate of the weighted coordinate positions Gᵢⱼₖ.

2. The pulse pressure measuring apparatus according to claim 1, wherein, in the blood vessel locus determination stage, each of the plurality of pressing elements simultaneously presses the site to be measured.

3. The pulse pressure measuring apparatus according to claim 1, wherein spacings of the adjacent pressing elements in a first direction and a second direction perpendicular to the first direction are less than or equal to 30 mm.

4. The pulse pressure measuring apparatus according to claim 1, wherein the band is defined based on the plurality of linear equations having a slope within ±0.5 relative to a baseline, and the blood vessel locus of the site to be measured corresponds to the band.

5. The pulse pressure measuring apparatus according to claim 4, wherein the blood vessel locus of the site to be measured is determined based on a barycentric coordinate of the plurality of weighted coordinate positions Gᵢⱼₖ and an average slope of the plurality of linear equations defining the band.

6. A pulse pressure measuring apparatus, including:
a base;
a plurality of pressing elements, arranged in an array on the base and used to press a site to be measured, wherein a number of the pressing elements is at least four, and each of the pressing elements has a position coordinate Pᵢ (i=1, 2, 3 . . . );
a plurality of pressure sensors, configured to respectively measure pressure on the plurality of pressing elements in order to generate measured values of pressure intensity Iᵢ (i=1, 2, 3 . . . ) at the position coordinates Pᵢ (i=1, 2, 3 . . . ); and
a processing unit, connected to the plurality of pressing elements and the plurality of pressure sensors,
wherein, in the blood vessel locus determination stage, the processing unit defines multiple virtual circles, wherein the position coordinates Pᵢ (i=1, 2, 3 . . . ) are respectively centers of the virtual circles and reciprocals of a square root of the measured values of pressure intensity Iᵢ (i=1, 2, 3 . . . ) are respectively radii of the virtual circles, and
the processing unit further defines a plurality of interior common tangents of the virtual circles, shrinks or enlarges the virtual circles by a same ratio until at least two of the interior common tangents overlap with each other, and defines a blood vessel locus of the site to be measured based on the interior common tangents overlapping with each other.

7. The pulse pressure measuring apparatus according to claim 6, wherein the overlapping interior common tangents defining the blood vessel locus of the site to be measured correspond to at least four of the position coordinates Pᵢ (i=1, 2, 3 . . . ).

8. The pulse pressure measuring apparatus according to claim 6, wherein the overlapping interior common tangents defining the blood vessel locus of the site to be measured respectively correspond to different pulse wave sources.

9. The pulse pressure measuring apparatus according to claim 6, wherein, in the blood vessel locus determination stage, each of the plurality of pressing elements simultaneously presses the site to be measured.

10. The pulse pressure measuring apparatus according to claim 6, wherein spacings of the adjacent pressing elements in a first direction and a second direction perpendicular to the first direction are less than or equal to 30 mm.
